(19) United States
(12) Patent Application Publication
(10) Pub. No.: US 2023/0044560 A1
(43) Pub. Date: Feb. 9, 2023

(54) METHOD FOR SEPARATION OF SPERM WITH UNDAMAGED INTACT HEADS FROM SPERM WITH DAMAGED HEADS AND SOMATIC CELLS

(71) Applicants: BIOTECHNOLOGICKY USTAV AV CR, V. V. I., Vestec (CZ); PRIMECELL BIOSCIENCE, A.S., Praha 1 (CZ); CESKA ZEMEDELSKA UNIVERZITA V PRAZE, Praha 6 (CZ)

(72) Inventors: Katerina KOMRSKOVA, Praha 4 (CZ); Pavla POSTLEROVA, Velke Prilepy (CZ); Michaela FROLIKOV A, Ricany (CZ); Serhiy FOROSTYAK, Praha 8 (CZ); Ondrej SIMONIK, Becvou (CZ)

(73) Assignees: BIOTECHNOLOGICKY USTAV AV CR, V. V. I., Vestec (CZ); PRIMECELL BIOSCIENCE, A.S., Praha 1 (CZ); CESKA ZEMEDELSKA UNIVERZITA V PRAZE, Praha 6 (CZ)

(21) Appl. No.: 17/792,566

(22) PCT Filed: Jan. 17, 2021
(86) PCT No.: PCT/CZ2021/050005

§ 371 Date: Jul. 13, 2022

(30) Foreign Application Priority Data
Jan. 17, 2020 (EP) 20152484.0

(51) Int. Cl. C12N 5/071; C07K 16/28

(52) U.S. Cl. CPC C12N 5/0612; C07K 16/2896

(57) ABSTRACT

A method of separation of sperm cells with undamaged intact heads from sperm cells with damaged heads and/or somatic cells and/or cell debris in a sperm sample obtained from a human, comprising the steps of:
providing an anti-CD46 antibody or CD46-binding ligand bound to a carrier and/or to a tag;
contacting the sperm sample with the anti-CD46 antibody or CD46 -binding ligand bound to the carrier and/or to the tag in order to bind CD46 presenting cells and/or cell debris from the sperm sample to the anti-CD46 antibody or CD46-binding ligand;
removing the CD46 presenting cells bound via the anti-CD46 antibody or CD46 -binding ligand to the carrier and/or to the tag to obtain a sperm sample free of CD46 presenting cells, wherein CD46 presenting cells include sperm cells with damaged heads, somatic cells and cell debris, is disclosed.

Method for separation of sperm with undamaged intact heads from sperm with damaged heads and somatic cells

### FIELD OF ART

The present invention relates to a method of separation of sperm with undamaged intact heads from sperm with damaged heads, as well as from somatic cells and cell debris, in cell-containing samples obtained from a human, such as ejaculate. This method enables, for example, to use the separated sperm with intact heads in assisted reproduction to enhance the fertility success.

### BACKGROUND ART

Artificial fertilization is a method widely used in the treatment of human reproductive disorders by means of assisted reproduction. In all applications of artificial fertilization, the sperm quality is of crucial importance for achieving high fertilization success rates. Technologies for improvement of sperm quality are therefore being developed and they are of high priority. Some of the available methods for sperm quality improvement rely on health regimens and/or dietary improvements implemented by the males. Other methods focus on improving of sperm quality by enriching the sample in healthy sperm. A representative of such method is the method described in US 2017/0191029 in which a sperm sample is contacted with an insoluble carrier, which binds to an indicator of impaired sperm membrane integrity or impaired sperm motility. However only commercially available beads were tested in this document, some of which allowed achieving approximately 20% increase in motility, where for example, anti-rabbit IgG seemed to improve the ratio of healthy sperm.

CD46 (membrane cofactor protein) is membrane-associated glycoprotein that is present in human cells including sperm and in cells of other mammals. In somatic cells, CD46 is present on their surface and it plays a key role in protecting host cells from complement attack—a mechanism which is also used by tumour cells. Another role of CD46 protein is activation of several intracellular pathways in different types of cells including T-lymphocytes. Several pathogens use it as a receptor enabling them to enter the cell. In sperm, CD46 is localized on the head, specifically in an acrosome, and it only becomes surface exposed after sperm acrosome reaction. The acrosome may be damaged, deformed or disrupted in non-physiological way. If the acrosome is damaged, the sperm is unable to penetrate the egg surroundings and fertilize the egg. The presence of damaged sperm, as well as somatic cells of any kind in the semen is not favourable for successful fertilization.

The role of CD46 in fertilization was already noticed by using monoclonal antibodies to the first short consensus repeat (SCR1) ectodomain of CD46, which resulted in blocking the complement-independent interaction of human sperm with zona-free oocytes/eggs in vitro. The recent discovery of a CD46 new human physiological ligand Jagged-1, which is highly expressed by the oocytes/eggs, strengthens the CD46 role in the initial steps of the sperm interaction with oolema (egg plasma membrane). CD46^{-/-} deficient mouse males show a higher rate of spontaneous acrosome reaction compared to wild type males. This finding led to the theory that CD46 plays a role in the stabilization of the acrosomal membrane and consequently the whole acrosome region. CD46 was reported to have a reduced expression in humans with idiopathic infertility.

In humans, the CD46 protein is expressed in all cells except erythrocytes and exists predominantly in four different isoforms—these isoforms are the products of alternative splicing of a single gene. They consist of four extracellular domains on the amino terminus called short consensus repeats (SCRs), a serine-threonine-proline (STP)-rich area, transmembrane domain (TM), intracytoplasmic anchor and one of two forms of cytoplasmic tail (CYT-1 of 16 amino acids and CYT-2 of 23 amino acids) that arise by alternative splicing at carboxyl terminus. The SCR1, 2 and 4 domains are sites of N-linked glycosylation. The STP region is a site of extensive O-linked glycosylation and is composed of 14 or 29 amino acids. The size of the STP region depends on the presence of STP exon B that can be possibly spliced out. Absence of STP exon B leads to lower molecular weight protein isoforms of CD46, while its presence leads to CD46 higher molecular weight isoforms. However, an even lower molecular weight hypoglycosylated isoform of CD46 is expressed in mammalian sperm. In humans, polymorphisms of CD46 gene were detected in normal individuals with both restriction enzymes, HindIII and EcoRI.

### Disclosure of the Invention

The present invention is a novel method for selection and separation of sperm with undamaged intact heads exploiting the fact that sperm with undamaged intact heads do not present CD46 on their surface in contrast to sperm with damaged heads (partially or totally damaged acrosomes) and majority of somatic cells, as well as cell debris, which present CD46 on their surface; and on a concept of using CD46 as a selector in a simple, fast and effective method.

The present invention provides a method of separation of sperm cells with undamaged intact heads from sperm cells with damaged heads and/or somatic cells and/or cell debris in a sperm sample obtained from a human, comprising the steps of:
providing an anti-CD46 antibody and/or a CD46-binding ligand bound to a carrier and/or to a tag,
contacting the sperm sample with the anti-CD46 antibody and/or the CD46-binding ligand bound to the carrier and/or to the tag in order to bind CD46 presenting cells and/or cell debris from the sperm sample to the anti-CD46 antibody and/or to the CD46-binding ligand,
removing the CD46 presenting cells bound via the anti-CD46 antibody and/or via the CD46-binding ligand to the carrier and/or to the tag to obtain a sperm sample free of CD46 presenting cells, wherein CD46 presenting cells include sperm cells with damaged heads, somatic cells and cell debris.

The present invention is based on utilization of the fact that the CD46 molecule (official full name provided by HGNC—HUGO Gene Nomenclature Committee; http://www.genenames.org/cgi-bin/gene_symbol_report?match=CD46 https://www.ncbi.nlm.nib.gov/gene/4179) is not presented on the surface of undamaged (undisrupted, intact) sperm heads. On the other hand, the sperm with damaged heads (in particular acrosomes) and somatic cells and cell debris present CD46 molecules on their surface. Thus, using anti-CD46 antibody or CD46-binding ligand as a selector, unbound sperm with undamaged intact healthy heads can be separated from the sperm marked or selected for damaged heads (namely acrosomes) as well as from somatic cells and debris that are present in an ejaculate. This allows to separate the undisrupted sperm with undamaged intact heads for further use, for example in assisted reproduction, and to remove the disrupted sperm with damaged heads, somatic cells and debris from a sperm sample.

The term “damaged sperm”, “sperm with damaged heads”, “sperm with disrupted heads” in this description is intended to designate sperm cells with partially or totally damaged heads, namely acrosomes, so that CD46 is presented on the surface of the sperm cells and/or is accessible for the antibody or CD46-binding ligands.

The term “sperm with undamaged intact heads” or “undisrupted sperm cells” or “undamaged sperm cells” or “sperm cells with undisrupted heads” or “sperm cells with undamaged heads” designates sperm cells with intact and undisrupted heads, so that CD46 is not presented on the surface of the sperm cells and/or is not accessible for the anti-CD46 antibody or CD46-binding ligands.

The term “cell debris” is characterized as cellular components and fragmented remnants of dead or damaged cells or tissues.

The term “sperm sample” includes a sample containing a mixture of cells including sperm cells. Preferably, the sperm sample is a sperm-containing sample such as an ejaculate, or a pre-treated sperm sample. The sperm sample used in the present invention is typically a sperm sample which has not yet been contacted with an egg, i.e., the sperm cells have not yet undergone the acrosome reaction.

The terms “binding” and “bound” include in particular conjugation, covalent binding or non-covalent binding.

The invention enables to separate sperm cells with undamaged intact heads from sperm cells labeled by anti-CD46 antibody or CD46-binding ligand for damaged heads (namely damaged acrosome), as well as from somatic cells and cell debris that are present in the sample. The sample may be ejaculate (or sperm), or a pre-treated ejaculate (or sperm). The pre-treatment may include removal of seminal fluid and/or tissues and/or debris from the ejaculate or from fresh or conserved sperm, e.g. by density gradient centrifugation or by a swim-up, or by any other suitable method.

The sample containing only sperm cells with undamaged intact head may further be used in assisted reproduction techniques, including in vitro fertilization, intracytoplasmic sperm injection etc.

The method of present invention may in some embodiments contain the following steps:
designing at least one immunization peptide for anti-CD46 antibody production or a peptide sequence of a CD46-binding ligand;
preparing an anti-CD46 antibody or a CD46-binding ligand;
binding the anti-CD46 antibody or the CD46-binding ligand to a carrier and/or to a tag;
contacting the anti-CD46 antibody or the CD46-binding ligand bound to the carrier and/or to the tag with a sperm sample in order to bind CD46 presenting cells, including sperm cells with damaged heads and/or somatic cells and/or cell debris;
separating the sperm cells with damaged heads and/or somatic cells and/or cell debris bound to the carrier and/or to the tag via the anti-CD46 antibody or the CD46-binding ligand from the sperm sample,
thereby obtaining two cell fractions: 1) sperm cells with undamaged intact heads and 2) sperm cells with damaged heads and/or somatic cells and/or cell debris.

The method thus results in a selection and separation of sperm cells with damaged heads and/or somatic cells and/or cell debris from the fraction of the sample not presenting CD46.

The “fraction of the sample not presenting CD46” or the “sperm sample free of CD46 presenting cells” are used herein as synonyms. This fraction of the sample may include sperm cells with undamaged intact head and/or acellular components of the sample and/or red blood cells.

Any obtained fraction, i.e., the fraction of the sample not presenting CD46, or the fraction of CD46-presenting cells bound to the carrier and/or to the tag, can be used for further processing as needed, for example in artificial fertilization technologies, medical applications, for storage purposes, or research.

The CD46 peptides for anti-CD46 antibody production can be the whole CD46 (molecule/protein/peptide/ligand/receptor) or its modifications, isoforms and transcript variants (synthetically prepared substances which contain/incorporate a part of the sequence of the molecule) and derivatives (synthetically prepared substances which constitute non-functional subunit of the molecule).

The anti-CD46 antibody may be a commercially available antibody or an antibody produced by known methods. The antibodies may be monoclonal or polyclonal, conjugated or unconjugated, directly or indirectly prepared antibodies against CD46. The anti-CD46 antibodies may be prepared by any suitable method, including, for example, proteomic synthesis, hybridoma cell line technology and immunization by a tissue, cells, whole protein, extracellular part, cytoplasmic tail or peptides with/without post-translational modifications. The anti-CD46 antibodies may also be or include synthetic antibodies or derivatives of synthetic antibodies.

For example, the anti-CD46 antibody may be prepared by a method including immunization of animals and/or cells by immunization peptides.

The CD46-binding ligand may be a commercially available peptide or a peptide produced by known methods. The peptides (with/without post-translational modifications) may be prepared by any suitable method, including, for example, proteomic synthesis, recombinant technology, production of ligands that bind to CD46 whole protein, extracellular part, cytoplasmic tail or any parts of the CD46 molecule. The CD46-binding ligand may also be or include synthetic peptides or peptide derivatives. One or more CD46-binding ligands are meant by reference to “a CD46-binding ligand”.

The carrier may be a substance or device capable of binding the anti-CD46 antibody or CD46-binding ligand or part thereof The carrier may preferably be in a solid form, in a form of solution or in a form of gel. Examples of suitable carriers include: beads, magnetic beads, polymeric substrates (e.g. polyacrylamide, polystyrene, polycarbonate), cellulosic substrates (e.g., agarose, sepharose), metallic substrates, magnetic or magnetizable carriers. Carriers are typically materials capable of covalent or non-covalent, specifically or non-specifically, directly or indirectly in single or multiple steps binding of the anti-CD46 antibody or CD46-binding ligand. The carriers may be applied by coating or immobilization on the surface of materials or devices. In some embodiments, the carriers or the antibodies or the binding ligands may be coated or immobilized on the inner surface of a vessel, such as a well, well plate, beaker, capillary or test tube (if antibodies or ligands are coated or immobilized on the inner surface of a vessel, then the vessel acts as a carrier).

The contacting step of anti-CD46 antibody or CD46-binding ligand with a carrier is preferably carried out at about room temperature, in a physiological buffer (such as phosphate buffer, physiological saline solution). The anti-CD46 antibody(ies) or CD46-binding ligand(s) bound to the carrier forms an anti-CD46 antibody-carrier complex or CD46-binding ligand-carrier complex.

The anti-CD46 antibody-carrier complex or CD46-binding ligand-carrier complex allows selection and separation of sperm cells with damaged heads and/or somatic cells and/or cell debris from sperm cells with undamaged intact heads from an ejaculate or from a sperm sample in general. Selection is based on presence or absence of CD46 labeling (meaning absence of CD46 labeling on the surface of sperm with undamaged intact heads).

The tag bound to the anti-CD46 antibody or CD46-binding ligand may be selected from fluorophores, chromophores, quantum dots, oligonucleotides, magnetic particles, coated magnetic particles (e.g., polymer-coated magnetic particles), His-tag, MBP-tag, GST-tag, CBP-tag and Strep-tag. Methods of tagging of antibodies are known in the art.

Magnetic particles and coated magnetic particles may preferably have the size from 1 to 3000 nm, more preferably from 1 to 500 nm, or from 1 to 200 nm.

In case of use of the anti-CD46 antibody-carrier complex or CD46-binding ligand-carrier complex, the removal of the CD46 presenting cells is typically based on the removal of the carrier to which the CD46 presenting cells are bound via the anti-CD46 antibody or CD46-binding ligand. In case of use of the tagged anti-CD46 antibody or tagged CD46-binding ligand (i.e., an anti-CD46 antibody or a CD46-binding ligand bound to a tag), the removal of the CD46 presenting cells is typically based on cell sorting based on the presence or absence of the tag.

The carrier separation techniques or separation techniques based on the presence or absence of the tag include methods capable to separate carrier comprising anti-CD46 antibody or CD46-binding ligand with or without the CD46 presenting cells and methods capable to separate tagged cells from untagged cells. The method may be direct/indirect, passive/active, gradient, biological, biochemical, physical, chemical, enzymatic, non-enzymatic, mechanical. The method may preferably be selected from centrifugation, sedimentation, flow separation methods such as FACS, capillary flow separation, separation on chips, or methods using a magnetic force in case of magnetic or magnetizable carriers and/or tags, for example MACS or magnetic separation. The removal step may also be performed by collecting the fraction of the sample containing the unbound sperm with undamaged intact heads, for example when the carrier is a vessel.

The method of the present invention is performed in vitro or ex vivo.

### BRIEF DESCRIPTION OF FIGURES

FIG. 1: Schematic model of a sperm: (A) sperm with an undamaged intact head and localization of CD46 inside the intact head, which is not accessible for the antibody nor CD46-binding ligand. (B, C) sperm with the damaged head, wherein CD46 can be detected by a species specific antibody or CD46-binding ligand. (B) a partial membrane damage and partial acrosome damage. (C) an excessive head damage with a loss of acrosome.

FIG. 2: CD46 detection of damaged sperm, somatic cells and cell debris using fluorescent microscope. Top left panel: anti-CD46 antibody labels sperm cells with damaged heads (arrows) and somatic cells (*) and cell debris (**). Sperm cells with intact undamaged heads (+) are not labeled by anti-CD46 antibody. Top right panel: all present sperm, somatic cells and debris labeled by Hoechst. Bottom left panel: Sperm cells with intact undamaged heads are not labeled by anti-CD46 antibody (+). Bottom right panel: Sperm with intact undamaged heads labeled by Hoechst.

FIG. 3: CD46 detection of damaged sperm, somatic cells and cell debris using Flow Cytometer. Left panel: population of cells after gating (marked by polygon). Middle panel: visualization of selected singlets. Right panel: Sperm with intact undamaged heads are not labeled by anti-CD46 antibody (−). Sperm with damaged heads are labeled with anti-CD46 antibody (+).

FIG. 4: Schematic model of sperm magnetic selection example: Ejaculate sample incubation with an antibody against CD46 conjugated to a magnetic carrier. Damaged sperm and non-spermatic cells bound to the carrier via the antibody are attracted to the magnet. Intact sperm are simply transferred by a pipette and ready for further utilization.

FIG. 5: Sperm sample quantitative evaluation before and after selection using anti-CD46 antibody examined for acrosome integrity and damage. Sperm sample before selection contains 75% sperm with damaged acrosomes and 25% sperm with intact acrosome. Intact sperm samples after magnetic selection using CD46 marker contains 18% sperm with acrosome damage and 82% of sperm with intact undamaged heads. N(samples)=10, error bars represent standard error of the mean (SEM).

FIG. 6: Sperm sample quantitative evaluation before and after selection using anti-CD46 antibody examined for total and progressive motility by Computer Assisted Sperm Analysis (CASA). Sperm sample before selection contains 32% sperm displaying total motility, 30% of sperm with progressive motility, which in total represent 62% of motile sperm. Intact sperm samples after selection using CD46 marker contains 48% sperm detected with total motility and 44% of sperm with progressive motility, which in total represent 92% of motile sperm. N(samples)=10, error bars represents standard error of the mean (SEM).

FIG. 7: Sperm sample quantitative evaluation after selection using anti-CD46 antibody examined for DNA integrity by TUNEL assay in fraction with damaged sperm and fraction with intact sperm. Damaged sperm sample contains 73% sperm positive for DNA fragmentation and 27% of sperm negative for DNA fragmentation. Intact sperm samples after selection using CD46 marker contains 13% sperm positive for DNA fragmentation and 87% sperm negative DNA fragmentation showing no detectable DNA damaged. N(samples)=10, error bars represents standard error of the mean (SEM).

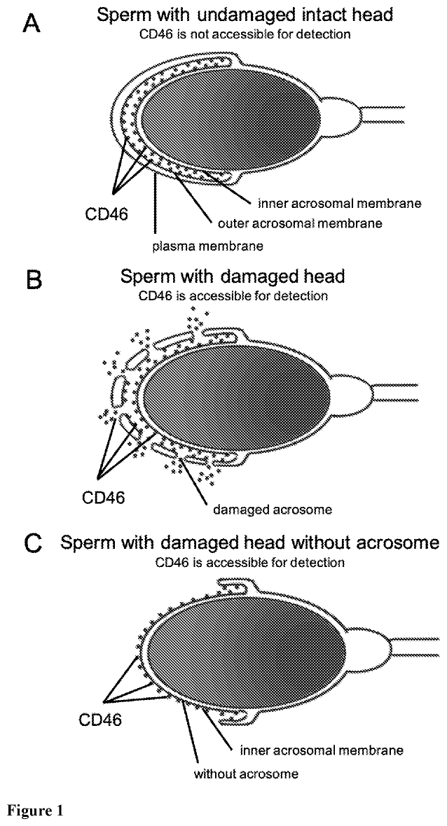

### EXAMPLES OF CARRYING OUT THE INVENTION

### Example 1: Differentiation of Sperm with Undamaged Intact Heads from Sperm with Damaged Heads, Somatic Cells and Cell Debris

Sperm cells in fresh human ejaculate were labeled for nucleus by Hoechst, and for the head integrity by an anti-CD46 primary antibody (clone M177, sc-52647, Santa Cruz Biotechnology, Inc.) visualized by a secondary fluorescent antibody (anti-mouse Alexa Fluor 488, Molecular Probes, Prague, Czech Republic). The Hoechst labeling showed all the cells present in the sample, while the labeling by anti-CD46 antibody showed only sperm cells with damaged head.

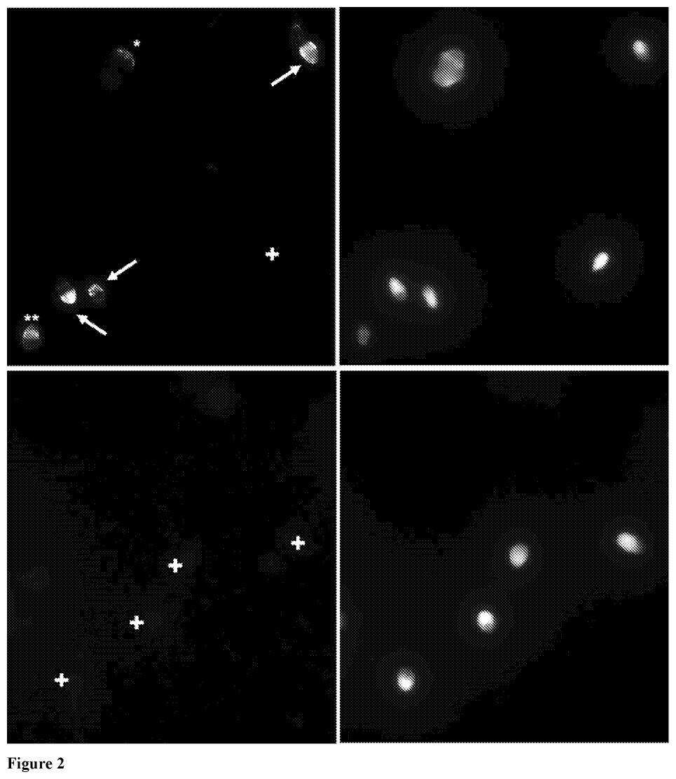

FIG. 2 shows an image obtained from the sample—Top left panel: anti-CD46 antibody labels sperm cells with damaged heads (arrows) and somatic cells (*) and cell debris (**). Top right panel: all present sperm, somatic cells and debris labeled by Hoechst. Bottom left panel: Sperm cells with intact undamaged heads are not labeled by anti-CD46 antibody (+). Bottom right panel: Sperm with intact undamaged heads labeled by Hoechst. The position of the sperm cell with undamaged intact head has been marked by a cross in the top and bottom left panels, for better understanding of the images by the reader.

### Example 2: Preparation of Monoclonal Anti-Human CD46 Antibody

A monoclonal anti-human CD46 antibody was prepared as follows:

The monoclonal anti-human CD46 antibody was prepared after immunization of mice (BALB/c strain) with 50 μg of the human CD46 peptide (TFSEVEVFEYLDAVTYS) conjugated with KLH (KLH=keyhole limpet hemocyanin) in 50 μl of phosphate buffer and 50 μl of Freund's adjuvant, subcutaneously in two doses over 10 days. Reimmunization has taken place with 200 μg KLH-peptide in 200 μl intraperitoneally for another 10 days, followed by fusion of spleen cells with myeloma cells after 3 days.

### Example 3: Magnetic Particle Synthesis

Maghemite nanoparticles size 150 nm were prepared by iron nitrate (Fe(NO₃)_{3.9}H₂O) reduction in the presence of the reducing agent, sodium borohydride. ₈ g of Fe(NO₃)_{3.9}H₂O was dissolved in 500 ml of water under continuous heating (100° C.) and stirring (600 rpm). While stirring, 1 g of NaBH₄ was added, previously dissolved in 50 ml of 3.5% NH₃. Then the obtained solution was heated at boiling temperature (130-150° C.) for 2.5 h under 900 rpm continuous stirring on magnetic stirrer. After cooling, the product was separated by external magnetic field and washed several times with dH₂O. The prepared magnetic particles were used as core for further surface modification.

### Example 4: Synthesis of Magnetic Particles having Average Size 35 nm

25 g of FeCl₃.6H₂O and 17 g of FeSO₄.7H₂O was taken in a 500 ml flask. Then 200 mL of 0.01 M HCl solutions was added and the solution mixed gently with magnetic stirrer (1000 rpm). After 10 min the whole mixture was heated on hot plate at 85° C. for 30 min and 60 ml ammonia solutions were added to the mixture and Oleic acid (4.6 mL) added immediately. The reaction was carried out at 85° C. temperature for 2 hrs. The flask was taken out from the magnetic hot plate for cooling and precipitates were washed with 500 mL of deionized water under magnetic stirring (1000 rpm) for 2 hrs. Synthetized magnetic nanoparticles were separated by magnetic decantation.

### Example 5: Surface Modification of Magnetic Particles

For surface modification of magnetic particles, polyethylene glycol (PEG) with molecular weight 6000, polyvinylpyrrolidone (PVP) with molecular weight 10,000 and dextran were used. PEG, PVP and dextran were dissolved (25% weight by volume) in PBS and added to magnetic particles at 1 ml/1 mg concentration. The mixture was placed in rotator for 24 hr rotation cycle at 37° C. temperature under continuous rotation and shaking. After rotation cycle the particles coated with modifying agent were separated by external magnetic field, and washed several times with dH2O, and kept in fridge for further

### Example 6: Antibody Coating

CD46 antibody was diluted in PBS in 20 μg/mL concentration for antibody coating on magnetic particles. The coated particles were dispersed in antibody solution 1 mg/1 mL concentration. The solution was further placed in 37° C. with continuous rotation and shaking overnight. The final antibody coated particles were separated by external magnetic field and washed three times with dH2O, dispersed in PBS and kept in 4° C. for further use.

### Example 7: Synthesis of Quantum Dots (QD)

Aqueous CdTe QDs were prepared as follows: 0.05 mL of 100 mM CdCl₂ was placed in a conical flask first, then 5.25 mL of water, 0.25 mL of 40 mM mercaptopropionic acid (MPA), 0.25 mL of 4 mM Na₂TeO₃, 0.6 mg of NaBH₄, and 34.2 mL of 85% N₂H₄ 3H₂O were added one after the other. The mixture was stored at room temperature to maintain the growth of QDs. After 2 h storage, the QDs with red emission were obtained. By altering the ratio of reagents, QDs with different emission colors were prepared. Meanwhile, the precipitates of QDs are gained by adding 2-propanol to QD solution and centrifugation.

### Example 8: Synthesis of Quantum Dots (QD)

Cadmium (Cd) precursor was prepared by mixing water, ethanol, and oleic acid together in volume ratio of 15: 35: 12, then 0.001 mol cadmium acetate and 0.7 g sodium hydroxide were added in 48 mL of the mixed solvent. The reaction temperature was 80° C. Selenium (Se) precursor was prepared by mixing the 0.005 mol selenium powder, 0.01 mol sodium sulfite, and 0.01 mol sodium hydroxide, in 50 mL deionized water under continuous magnetic stirring. The mixture was heated to 130° C. then 0.01 mol sodium hydroxide was added, and the reaction system was kept under stirring and heating for 3 hours. After adequate reaction time the reactants were cooled down to room temperature. The CdSe QDs were synthesized by adding 10 mL Se precursor solution to the 48 mL Cd precursor solution, under magnetic stirring. The reactions were carried out at 55° C., 80° C., and 95° C. for 3 min, 8 min, 12 min, 15 min, and 20 min. The samples were centrifuged with ethanol and dispersed in hexane.

QD Capping: The capping was done with mercaptopropionic acid and triethylamine at 40° C. while magnetic stirring. A CD46-binding ligand or peptide is added for further use.

### Example 9: General Procedure for Separation of Sperm with Undamaged Intact Heads from Sperm with Damaged Heads from Human Ejaculate

Material: Cell sample suspension (human ejaculate), primary anti-human antibody anti-CD46 (if not specified otherwise, sc-52647) conjugated to a carrier.

Method: After the liquefaction of ejaculate without using any separation method, the ejaculate is incubated for 30 minutes with the anti-CD46 antibody conjugated to the carrier at room temperature, in a physiological buffer (PBS, phosphate buffer saline). Standard procedures and materials were used for binding the antibody to the carrier. The incubation of the anti-CD46 antibody-carrier complex with the ejaculate was followed by separation of the carrier with the conjugated anti-CD46 antibody to which the CD46 presenting cells are bound. The remaining unbound sperm fraction with undamaged intact heads was transferred to a new container for further use in assisted reproduction such as intrauterine insemination (IUI), in vitro fertilization (WF) or intracytoplasmic sperm injection (ICSI), storage purpose, medicine, or any other technique or research.

### Example 10: Separation of Sperm with Undamaged Intact Heads from Sperm with Damaged Heads from Human Ejaculate

The anti-human CD46 antibody is bound to magnetic beads as a carrier to form an anti-human CD46 antibody-carrier complex. The procedure of Example 9 was then followed. All the CD46 presenting sperm with damaged heads and somatic cells, mainly white blood cells, and cell debris are bound to the anti-human CD46 antibody-carrier complex and further separated by magnetic field, using a magnetic stand. The unbound sperm with undamaged intact heads remains in the suspension.

### Example 11: Separation of Sperm with Undamaged Intact Heads from Sperm with Damaged Heads for Storage Purpose

The anti-human CD46 antibody is bound to polyacrylamide beads as a carrier to form an anti-human CD46 antibody-carrier complex. The procedure of Example 9 was then followed. All the CD46 presenting sperm with damaged heads and all somatic cells, mainly white blood cells, and cell debris are bound to the carrier via the anti-CD46 antibody, and further removed from the ejaculate by centrifugation. The unbound sperm with undamaged intact heads remain in the suspension and they are ready to be used for short or long-term storage such as cryopreservation or any kind of sample preservation.

### Example 12: General Procedure for Separation of Sperm with Undamaged Intact Heads from Sperm with Damaged Heads from Cryopreserved Human Ejaculate

Material: Cryopreserved sample (human ejaculate), primary anti-human antibody anti-CD46 (if not specified otherwise, sc-52647) conjugated to a carrier or conjugated with a tag.

Method: After a sample thawing, the sample is incubated for 30 minutes with the anti-CD46 antibody conjugated to the carrier or conjugated with the tag, at room temperature, in a physiological buffer. Standard procedures and materials were used for binding the antibody to the carrier or to the tag. The incubation of the anti-CD46 antibody-carrier complex or anti-CD46 antibody-tag with the ejaculate was followed by separation of the carrier or tag with the conjugated anti-CD46 antibody to which the CD46 presenting cells are bound. The remaining unbound sperm fraction with undamaged intact heads is transferred to a new container for further use in assisted reproduction (artificial insemination) such as intrauterine insemination (IUI), in vitro fertilization (WF) or intracytoplasmic sperm injection (ICSI), storage purpose, medicine, or any other technique or research.

### Example 13: Separation of Sperm with Undamaged Intact Heads from Sperm with Damaged Heads from Cryopreserved Sperm Sample

The procedure of Example 12 was followed, wherein the carrier are magnetic beads. All the CD46 presenting sperm with damaged heads and all somatic cells, mainly white blood cells, and cell debris are bound to the anti-human CD46 antibody-carrier complex and further separated by magnetic field. The unbound sperm with undamaged intact heads remain in the suspension.

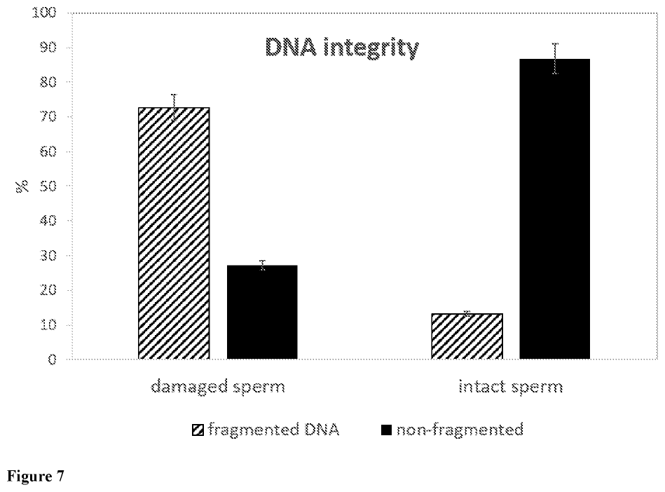

### Example 14: Sperm Motility Analysis (FIG. 6)

Material: Sperm sample after thawing (before selection) or after selection.

Method: Motility was measured with a Computer Assisted Sperm Analysis (CASA) system (ISAS, Proiser, Valencia, Spain). Total motility and progressive motility were evaluated based on manufacturer's setting (thresholds were VAP 10 um.s⁻and STR 80% for motile and progressively motile sperm, respectively). A 2 μL drop of each semen sample was placed in a 37° C. prewarmed Leja counting chamber (IMV Technologies, France). Six fields per sample were evaluated at 100× magnification negative phase-contrast objective. Constant temperature during analysis was ensured by a heating plate prewarmed to 37° C. as a part of microscope. The evaluation was based on the analysis of 60 consecutive digitized images, which were taken at a time lapse of 1 s with a camera at a frequency of 60 fps. At least 100 trajectories were analysed per field.

### Example 15: Sperm Acrosomal Damage Detection by CD46 Antibody with Fluorescent Tag Using Fluorescent Microscopy (FIG. 2)

Material: Sperm sample after thawing (before selection) or after selection.

Method: Sperm samples were washed twice (300 g, 5 min) in PBS. Pellet was diluted in 100 μL of PBS buffer and 10 μL of mouse monoclonal anti-CD46 antibody (clone M177, ThermoFisher Scientific, USA) conjugated with fluorescence tag (FITC). After 30 minutes incubation in room temperature, spermatozoa were washed in PBS. 9 μL of sperm suspension were transferred onto slide and gently mixed with 3 μL of Vectashield/DAPI (Vector Laboratories Ltd., Peterborough, UK) and mounted under a coverslip. Images were captured in magnification 600× by camera as part of epifluorescent microscope Nikon Eclipse E600 (Tokyo, Japan) and analysed by software NIS Elements (Laboratory Imaging, Inc., Prague, Czech Republic).

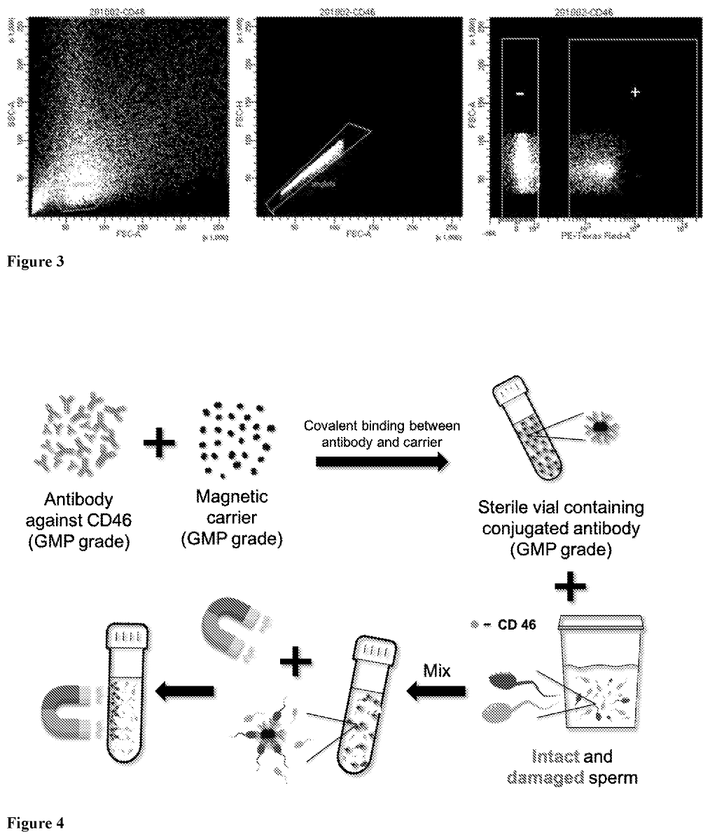

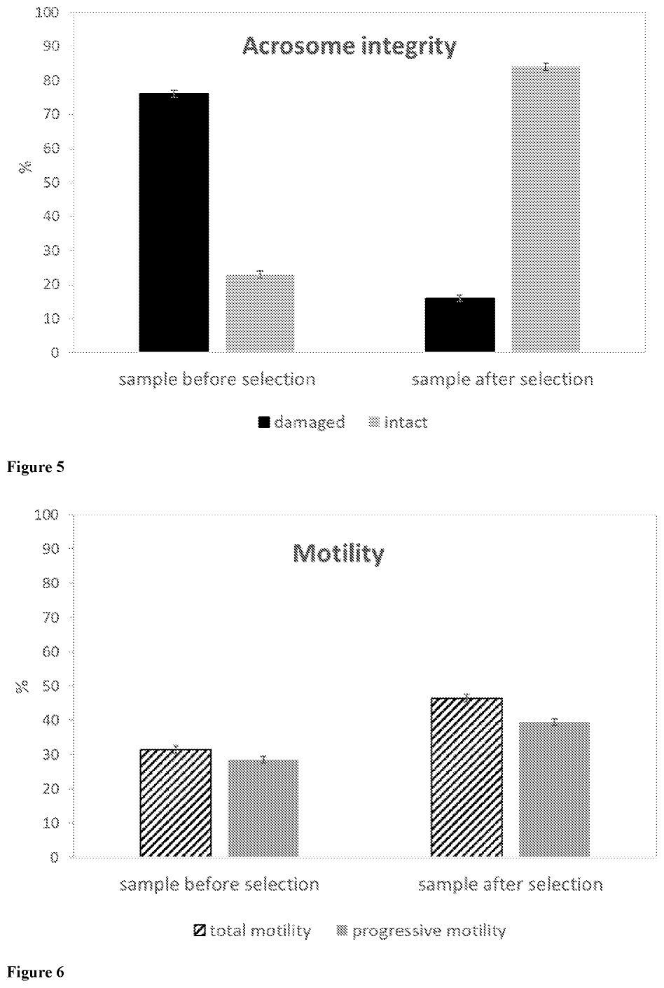

### Example 16: Sperm Acrosomal Damage Examined by CD46 Antibody Using Flow Cytometry (FIG. 3)

Material: Sperm sample after thawing or native liquified ejaculate.

Method: Sperm samples were washed twice (300 g, 5 min) in PBS. Sperm concentration was adjusted by PBS to 5−10×10⁶/mL. 10 μL of mouse monoclonal anti-CD46 antibody (clone M177, ThermoFisher Scientific, USA) conjugated with fluorescence tag (TRITC) was added into 100 μL aliquot. After 30 minutes incubation in room temperature, spermatozoa were washed twice in PBS. Sperm pellet was resuspended in 500 μL of PBS and samples were analysed by BD LSR Fortessa™ SORP (BD Biosciences, San Jose, Calif., USA). Based on fluorescence signal sperm was divided to CD46+ (Acrosome damaged) and CD46− (Acrosome intact).

Example 17: Sperm DNA Integrity Analysis (FIG. 7)

Material: Sperm sample after thawing sorted for damaged (CD46 positive) and intact (CD46 negative) sperm fraction.

Method: For sperm DNA integrity analysis (TUNEL) Apo-direct Kit (Phoenix Flow System, USA) was used. Spermatozoa were washed two times 250 ×gfor 5 min. Afterwards, 1 mL of Intracellular Perm Wash Buffer (Biolegend, San Diego, USA) was added to the pellet and suspension was incubated for 30 min in 4° C., centrifuged and washed two times. Sperm pellet was fixed in 1 mL of ice-cold 70% ethanol, vortexed and incubated for 30 min in 4° C. After fixation samples were centrifuged 300 ×g, 10 min, vortexed and washed two times (300 ×g, 10 minutes) in Wash buffer. Thereafter, a reaction mix was added for 60 min incubation in 37° C. Samples were washed twice with Rinse Buffer (300 ×g5 min) and 0.5 mL of Propidium iodide/RNAse A free was added for 30 min and samples were proceeded for analysis. Samples were analysed by flow cytometer BD LSR Fortessa™ SORP, BD Biosciences, San Jose, Calif., USA. For each sample at least 15000 events were recorded.

### INDUSTRIAL APPLICABILITY

The present invention is a technology for selection and separation of CD46 presenting components from cells not presenting CD46 in cell samples. The cells not presenting CD46 are sperm with undamaged intact heads. The technology may thus serve for separation of sperm with undamaged intact heads from sperm with damaged heads, as well as from somatic cells and cell debris in a human sperm sample. This technology is designed to separate sperm with undamaged intact heads to be further used in assisted reproduction, human medicine and research. The invention results in an increased quality of the sperm cell sample, as well as allows separation of somatic cells and/or debris from sperm-containing samples. Such purified samples result in an increased fertilization or medical or research success. The invention also results in an increased quality of the sperm sample for long- or short-term storage under any condition and media such as cryopreservation. This invention also saves large amount of cryoprotectives and plastic by storage of the pre-selected quality sample.

1. A method of separation of sperm cells with undamaged intact heads from sperm cells with damaged heads and/or somatic cells and/or cell debris in a sperm sample obtained from a human, comprising the steps of:
providing an anti-CD46 antibody or a CD46-binding ligand bound to a carrier and/or to a tag;
contacting the sperm sample with the anti-CD46 antibody or the CD46-binding ligand bound to the carrier and/or to the tag in order to bind CD46 presenting cells and/or cell debris from the sperm sample to the anti-CD46 antibody or the CD46-binding ligand;
removing the CD46 presenting cells bound via the anti-CD46 antibody or CD46-binding ligand to the carrier and/or to the tag to obtain a sperm sample free of CD46 presenting cells, wherein CD46 presenting cells include sperm cells with damaged heads, somatic cells and cell debris.

2. The method according to claim 1, wherein the sperm sample free of CD46 presenting cells is a sperm containing substantially only sperm cells with undamaged intact heads.

3. The method according to claim 1, wherein the starting sperm sample is an ejaculate or a pre-treated ejaculate, wherein the pre-treatment preferably includes removal of seminal fluid and/or tissues and/or cell debris from the ejaculate.

4. The method according to claim 1, which contains the following steps:
providing at least one immunization peptide for anti-CD46 antibody production or a peptide sequence of a CD46-binding ligand;
preparing an anti-CD46 antibody or a CD46-binding ligand;
binding the anti-CD46 antibody or the CD46-binding ligand to a carrier and/or to a tag;
contacting the anti-CD46 antibody or the CD46-binding ligand bound to the carrier and/or to the tag with a sperm sample in order to bind CD46 presenting cells, including sperm cells with damaged heads and/or somatic cells and/or cell debris;
separating the sperm cells with damaged heads and/or somatic cells and/or cell debris bound to the carrier and/or to the tag via the anti-CD46 antibody or the CD46-binding ligand from the sperm sample,
thereby obtaining two cell fractions: 1) sperm cells with undamaged intact heads and 2) sperm cells with damaged heads and/or somatic cells and/or cell debris.

5. The method according to claim 1, wherein the carrier is selected from the group comprising beads, magnetic beads, polymeric substrates, cellulosic substrates, metallic substrates, magnetic or magnetizable carriers.

6. The method according to claim 1, wherein the carrier is a vessel, such as a well, well plate, beaker, capillary or test tube, wherein the anti-CD46 antibody or CD46-binding ligand is coated or immobilized on the inner surface of the vessel; or wherein the carrier is coated or immobilized on the inner surface of a vessel, such as a well, well plate, beaker, capillary or test tube.

7. The method according to claim 1, wherein the removal of the CD46 presenting cells bound via anti-CD46 antibody or CD46-binding ligand to the carrier and/or to the tag is carried out by centrifugation, by sedimentation, by flow separation methods such as FACS, capillary flow separation, separation on chips, or by use of magnetic force such as MACS or magnetic separation.

8. A method of in vitro separation of sperm cells with undamaged intact heads from sperm cells with damaged heads and/or somatic cells and/or cell debris in a sperm sample obtained from a human, comprising the step of employing an anti-CD46 antibody or CD46-binding ligand.


SEQUENCE LISTING (1 sequence)

SEQ ID NO: 1 - protein, 17 aa, artificial
  immunization peptide
TFSEVEVFEYLDAVTYS